(19) United States
(12) Patent Application Publication
(10) Pub. No.: US 2004/0215282 A1
(43) Pub. Date: Oct. 28, 2004

(54) Connector module designs for implantable medical devices

(76) Inventors: Hans Van Der Weijden, Ermelo (NL); Freek Schurr, Utrecht (NL); Bert Van Wilsem, Steenderen (NL); Marc W.J.G Ettema, Enschede (NL); Paul Roelofsen, Arnhem (NL); Hennie Soethof, Glesbeek (NL); Murk Hofstra, Dieren (NL)

Correspondence Address: MEDTRONIC, INC., 710 MEDTRONIC PARKWAY NE, MS-LC340, MINNEAPOLIS, MN, 55432-5604, US

(21) Appl. No.: 10/422,081

(22) Filed: Apr. 23, 2003

(51) Int. Cl. A61N 1/375

(52) U.S. Cl. 607/37

(57) ABSTRACT

The invention is directed to connector module designs for facilitating electrical connection between a medical lead and circuitry of an implantable medical device (IMD). The connector module includes a channel for receiving a medical lead and one or more electrical contact elements are disposed along the channel to electrically couple to the lead when the lead is inserted into the channel. The connector module is formed with a hole to provide access to the channel, and a set screw can be disposed in the hole to secure the lead in place once it is inserted. A plug element covers the set screw and is formed with a slit that allows a tool to be inserted through the plug element to tighten the set screw. Moreover, a retention element retains the plug element in the connector module.

### FIELD OF THE INVENTION

The invention relates to implantable medical devices (IMDS) and, more particularly, to a connector module of an IMD that receives medical leads for coupling to the IMD.

### BACKGROUND OF THE INVENTION

In the medical field, leads are used with a wide variety of medical devices. For example, leads are commonly coupled to implantable cardiac pacemakers that provide therapeutic stimulation to the heart by delivering pacing, cardioversion or defibrillation pulses. The pulses can be delivered to the heart via electrodes disposed on the leads, e.g., typically near distal ends of the leads. In that case, the leads position the electrodes with respect to various cardiac locations so that the pacemaker can deliver pulses to the appropriate locations. Leads are also used for sensing purposes, or both sensing and stimulation purposes.

In addition, leads are used with neurological devices such as deep-brain stimulation devices, and spinal cord stimulation devices. For example, the leads can be stereotactically probed into the brain to position electrodes for deep brain stimulation. Leads are also used with a wide variety of other medical devices including, for example, devices that provide muscular stimulation therapy, devices that sense chemical conditions in a patient's blood, gastric system stimulators, implantable nerve stimulators, implantable lower colon stimulators, e.g., in graciloplasty applications, implantable drug or beneficial agent dispensers or pumps, implantable cardiac signal loops or other types of recorders or monitors, implantable gene therapy delivery devices, implantable incontinence prevention or monitoring devices, implantable insulin pumps or monitoring devices, implantable hearing restoration devices, and the like. In short, medical leads can be used for sensing purposes, stimulation purposes, drug delivery, and the like.

One challenge in implementing medical leads with an IMD is the electrical and mechanical coupling between a respective lead and the IMD. An IMD typically includes a housing that houses circuitry of the IMD, and a connector module that receives the lead and couples the lead to the circuitry. In particular, the connector module includes electrical contact structures for coupling the lead to circuitry within the housing of the IMD so that therapeutic simulation can be provided through the lead, or sensed conditions can be recorded by the circuitry.

Various connection standards have been developed in order to ensure electrical connections between the IMD circuitry and the lead are acceptable, while also maintaining a sufficient hermetic seal between the connector module and the lead to avoid ingress of body fluids into the housing. These standards continue to evolve to accommodate new lead designs, such as in-line leads that include a plurality of electrical contact areas along axial positions of the lead. The IS-1 and IS-4 standards are two examples of connection standards for cardiac pacemakers.

In general, there remains a need for improvements in connector modules of IMDs in order to simplify a physician's task of attaching leads to the IMD and ensure a good hermetic seal. In addition, cost reduction issues exist with respect to connector module designs and fabrication. Improved simplicity can help ensure that physicians can make the electrical connections during implantation of the IMD with minimal concern for electrical coupling malfunction. Reduced fabrication expense can help ensure that patient costs associated with an IMD are minimized.

### BRIEF SUMMARY OF THE INVENTION

In general, the invention is directed to connector module designs for facilitating electrical coupling between a medical lead and circuitry of an implantable medical device (IMD). The connector module includes a channel for receiving a medical lead and one or more electrical contact elements disposed along the channel to electrically couple to the lead when the lead is inserted into the channel. The electrical contact elements, in turn, electrically couple to the circuitry within the IMD.

The connector module is formed with a hole to provide access to the channel. A set screw can be disposed in the hole to secure the lead in place once it is inserted. In accordance with the invention, a plug element covers the set screw and is formed with a slit that allows a tool to be inserted through the plug element to tighten the set screw. Moreover, a retention element retains the plug element in the connector module. The invention can eliminate the need for an adhesive or silicone seal over the hole, thereby simplifying the process of connecting a lead to the connector module. Moreover, the invention can reduce costs associated with fabrication of an IMD and can facilitate large scale production of the various elements of the IMD connector module.

In one embodiment, the invention provides an implantable medical device comprising a housing, circuitry within the housing, and a connector module to receive a medical lead and electrically couple the medical lead to the circuitry. The connector module includes a channel to receive the lead, an electrical contact element disposed along the channel to electrically couple to the lead, a hole formed in the connector module for access to the channel, a set screw in the hole to hold the lead in place following insertion of the lead into the channel, a plug element in the hole covering the set screw and formed with a slit that allows a tool to be inserted through the plug element to tighten the set screw, and a retention element to retain the plug element in the connector module.

In another embodiment, the invention provides a connector module of an implantable medical device to receive a medical lead and electrically couple the medical lead to circuitry of the implantable medical device. The connector module comprises a channel to receive the lead, an electrical contact element disposed along the channel to electrically couple to the lead, a hole formed in the connector module for access to the channel, a set screw in the hole to hold the lead in place following insertion of the lead through the channel, a plug element in the hole covering the set screw and formed with a slit that allows a tool to be inserted through the plug element to tighten the set screw, and a retention element to retain the plug element in the connector module.

In another embodiment, the invention provides an implantable medical device comprising a housing, circuitry within the housing, and a connector module to receive a medical lead and electrically couple the medical lead to the circuitry. The connector module includes a channel to receive the lead, an electrical contact element disposed along the channel to electrically couple to the lead, a hole formed in the connector module for access to the channel, a set screw in the hole to hold the lead in place following insertion of the lead through the channel, a plug element in the hole covering the set screw and formed with a slit that allows a tool to be inserted through the plug element to tighten the set screw, and means for retaining the plug element in the connector module without the use of an adhesive.

In another embodiment, the invention provides a method comprising inserting a set screw in the hole of a connector module of an implantable medical device such that the set screw can be tightened to secure a lead in a channel of the connector module, inserting a plug in the hole over the set screw, the plug being formed with a slit to allow access to the set screw, and ultrasonically welding a retention element over the plug to retain the plug in the hole.

### BRIEF DESCRIPTION OF THE DRAWINGS

FIG. 1 is a perspective view of a system that includes an implantable medical device (IMD) according to an embodiment of the invention, and one or more medical leads coupled to IMD.

FIGS. 2-10 are cross sectional side views of portions of connector modules of IMDs according to embodiments of the invention.

FIG. 11 is a flow diagram illustrating the assembly and use of an IMD according to an embodiment of the invention.

### DETAILED DESCRIPTION OF THE INVENTION

The invention is directed to connector module designs for facilitating electrical connection between a medical lead and circuitry of an implantable medical device (IMD). The connector module includes a channel for receiving a medical lead and one or more electrical contact elements are disposed along the channel to electrically couple to the lead when the lead is inserted into the channel. The electrical contact elements, in turn, electrically couple to the circuitry within the IMD

The connector module is formed with a hole to provide access to the channel, and a set screw can be disposed in the hole to secure the lead in place once it is inserted. In accordance with the invention, a plug element covers the set screw and is formed with a slit that allows a tool to be inserted through the plug element to tighten the set screw. Moreover, a retention element retains the plug element in the connector module.

The invention can eliminate the need for an adhesive or silicone seal over the hole, thereby simplifying the process of connecting a lead to the connector module. Moreover, the invention can reduce costs associated with fabrication of an IMD and can facilitate large scale production of the various elements of the IMO connector module. The plug element can allow access to the set screw, e.g., via a slit in the plug element, but can still ensure a good hermetic barrier to avoid the ingress of fluids into the connector module, once the IMD is implanted in a patient. The retention element can ensure that the plug element is retained in the hole and thereby ensure that the hermetic barrier is maintained at all times.

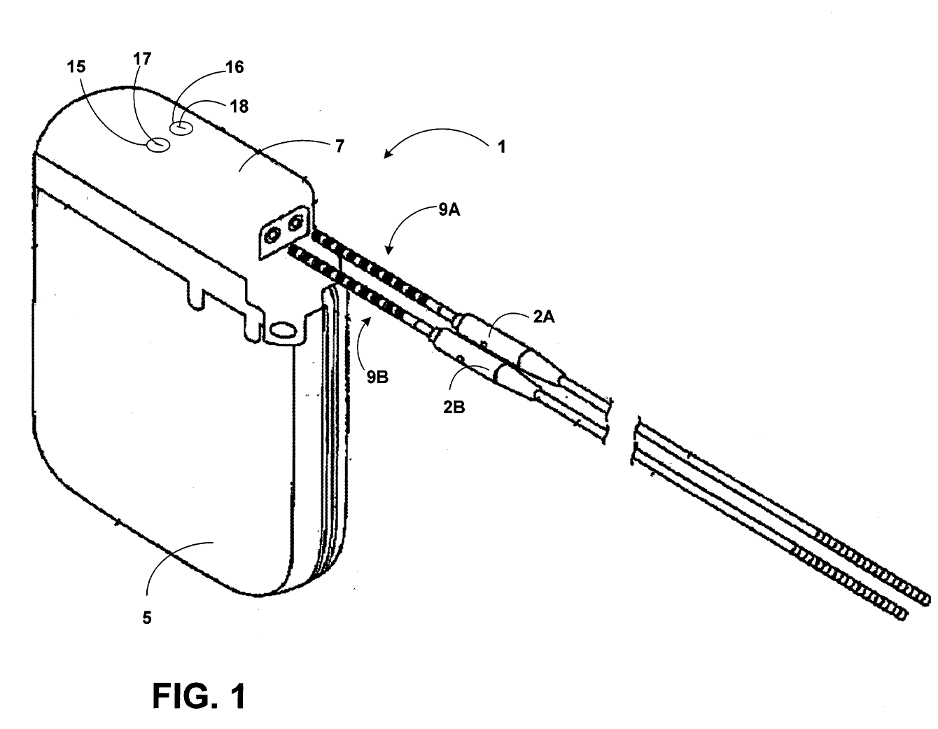

FIG. 1 is a perspective view of a system 10 that includes an IMD 12 and one or more medical leads 2A, 2B (collectively leads 2) coupled to IMD 12. IMD 12 includes a housing 5 that houses IMD circuitry and a connector module 7 that receives proximal ends 9A, 9B of leads 2 to couple leads 2 to the circuitry in housing 5. For example, connector module 7 may form part of housing 5, or can be viewed as a separate component that is mechanically attached to housing 5.

As illustrated in FIG. 1, the proximal ends 9A, 9B of leads 2 may include a plurality of a electrical contact areas arranged in an inline configuration. Connector module 7 facilitates electrical coupling to one or more of these contact areas. The contact areas of leads 2 may couple to electrodes of the leads via conductive filars that extend through leads 2, e.g., in a coiled construction.

Upon insertion of leads 2 into connector module 7, set screws (not shown) can be tightened to secure the leads. Connector module 7 includes plug elements 15, 16 which respectively cover holes where the set screws reside. Slits 17, 18 in plug elements 15, 16 allow for access to the set screws, e.g., via a tool that is inserted through slits 17, 18. In accordance with the invention, retention elements are used to retain plug elements 15, 16 within the respective holes and thereby ensure a good hermetic seal.

IMD 12 may correspond to any medical device that can receive medical leads. By way of example, IMD 12 may take the form of an implantable cardiac pacemaker that provides therapeutic stimulation to the heart. Alternatively, IMD 12 may take the form of an implantable cardioverter, an implantable defibrillator, or an implantable cardiac pacemaker-cardioverter-defibrillator. IMD 12 may deliver pacing, cardioversion or defibrillation pulses to a patient via electrodes disposed on distal ends of leads 2. In other words, leads 2 may position electrodes with respect to various cardiac locations so that IMD 12 can deliver pulses to the appropriate locations.

Alternatively, IMD 12 may correspond to a patient monitoring device, or a device that integrates monitoring and stimulation features. In those cases, leads 2 may include sensors disposed on distal ends of the respective lead for sensing patient conditions. The sensors may comprise electrical sensors, electrochemical sensors, pressure sensors, flow sensors, acoustic sensors, optical sensors, or the like. In many cases, IMD 12 can perform both sensing and stimulation functions.

In still other applications, IMD 12 may correspond to a neurological device such as a deep-brain stimulation device or a spinal cord stimulation device. In those cases, leads 2 may be stereotactically probed into the brain to position electrodes for deep brain stimulation or into the spine for spinal stimulation. In other applications, IMD 12 may provide muscular stimulation therapy, blood sensing functions, and the like. In short, IMD 12 may correspond to any of a wide variety of medical devices that implement leads and circuitry coupled to the leads.

As outlined in detail below, connector module 7 incorporates plug elements 15, 16 that allow for access to set screws. Moreover, connector module 7 makes use of non-adhesive retention elements to retain plug elements 15, 16. The described retention elements can improve retention of plug elements and facilitate large scale manufacturing of IMD 12, and can eliminate the need for a physician to apply adhesive over the access holes to the set screws.

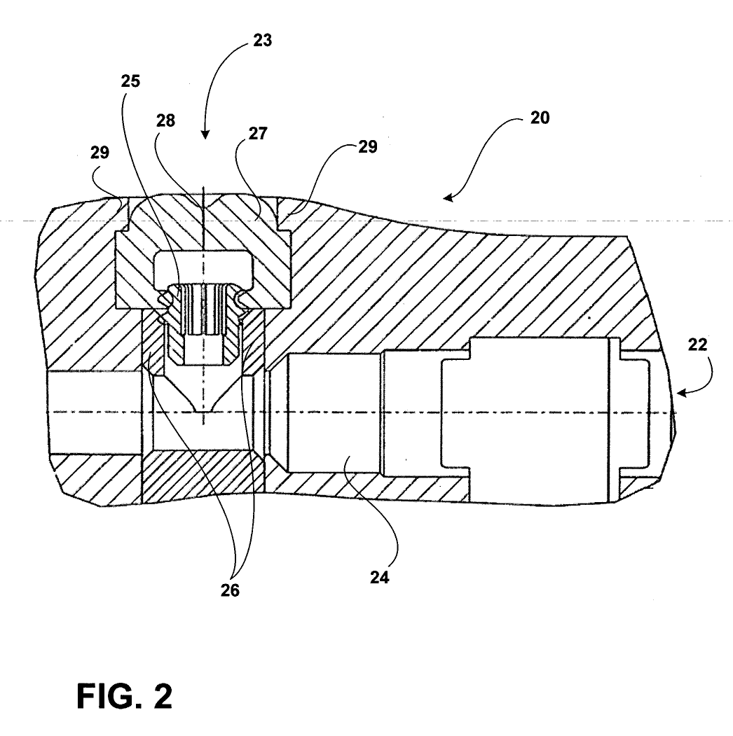

FIG. 2 is a cross-sectional side view of a portion of a connector module 20, that may correspond to connector module 7 of FIG.1. Connector module 20 includes a channel 22 sized to receive a proximal end of a medical lead 24. An electrically conductive element 26 is disposed along channel 22 to electrically couple to medical lead 24. Electrically conductive element 26 also couples to circuitry within the IMD. Connector module 20 is formed with a hole 23, e.g., perpendicular to channel 22. A set screw 25 is disposed in hole 23 to tighten lead 24 in place and ensure electrical coupling between lead 24 and electrically conductive element 26.

In accordance with the invention, a plug element 27 is positioned in hole 23 over set screw 25. In this example, plug element 27 is formed to mechanically interact with set screw 25, e.g., to fit over and around set screw 25. Plug element 27 defines a slit 28. A physician can insert a tool through slit 28 to tighten set screw 25. Upon removal of the tool from slit 28, however, slit 28 closes to provide a hermetic barrier to the interior of hole 23.

Connector module 20 further includes a retention element 29. In the example of FIG. 2, retention element 29 comprises an integrally formed lip of connector module 20 that serves to hold plug element 27 in place. Such a configuration illustrated in FIG. 2, however, can be very difficult to manufacture and assemble. A grinding process could be used to grind away an interior wall of hole 23 and thereby create retention element 29 in the form of a lip. Insertion of plug element 27 into hole 23, however, would still be difficult.

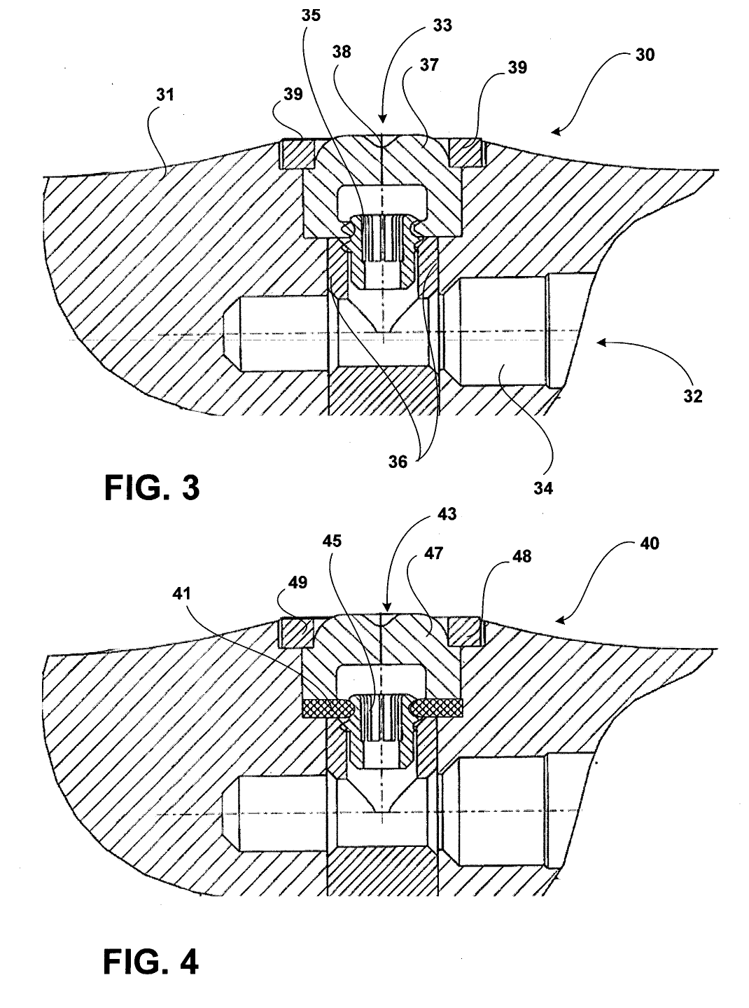

FIG. 3 is another cross sectional side view of a portion of a connector module 30, that may correspond to connector module 7 of FIG. 1. Connector module 30 includes a channel 32 sized to receive a proximal end of a medical lead 34. An electrically conductive element 36 is disposed along channel 32 to electrically couple to medical lead 34. Electrically conductive element 36 also couples to circuitry within the IMD Connector module 30 is formed with a hole 33, e.g., perpendicular to channel 32. A set screw 35 is disposed in hole 33 to tighten lead 34 in place and ensure electrical coupling between lead 34 and electrically conductive element 36.

A plug element 37 is positioned in hole 33 over set screw 35. In the example of FIG. 3, like that of FIG. 2, plug element 37 is formed to mechanically interact with set screw 35, e.g., to fit over the threading of set screw 35. Plug element 37 defines a slit 38. A physician can insert a tool through slit 38 to tighten set screw 35. Upon removal of the tool from slit 38, however, slit 38 closes to provide a hermetic barrier to the interior of hole 33.

Connector module 30 further includes a retention element 39. In the example of FIG. 3, retention element 39 comprises a ring element that is ultrasonically welded over hole 33 to secure and retain plug element 37. The retention element 39 may comprise a material that is the same as material 31 of connector module 30, e.g., a hard thermoplastic material. Ultrasonic welding generally melts retention element 39 to material 31. In this manner, fabrication of connector module 30 can be simplified and well suited for large scale production.

FIG. 4 is another cross sectional side view of a portion of a connector module 40, that may correspond to connector module 7 of FIG. 1. Connector module 40 is substantially similar to connector module 30 of FIG. 3. However, plug element 47 of connector module 40 is not formed to mechanically interact with set screw 45. Instead, an additional metal or synthetic ring 41 is provided to improve mechanical interaction and prevent unlock of set screw 45 during transport. In addition, metal or synthetic ring 41 can provide an additional barrier between set screw 45 and the lead. Set screw 45 generally passes through ring 41 but typically does not mechanically thread into ring. Plug element 47 is positioned over set screw 45 and metal or synthetic ring 41, and is retained by retention element 49. For example, retention element 49 may comprise a ring element that is ultrasonically welded over hole 43 to secure and retain plug element 47.

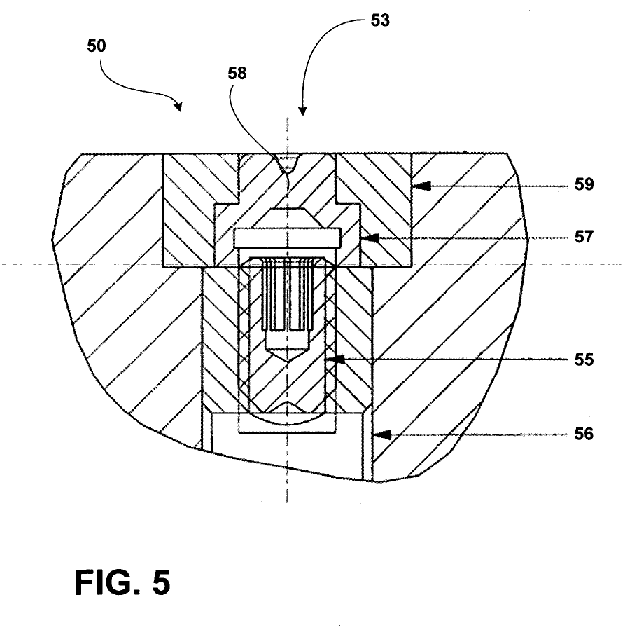

FIG. 5 is another cross sectional side view of a portion of a connector module 50, that may correspond to connector module 7 of FIG. 1. Connector module 50 includes a channel (not shown) sized to receive a proximal end of a medical lead (not shown). Upon insertion of the medical lead into the channel, the medical lead comes into electrical contact with electrically conductive element 56. Electrically conductive element 56 also couples to circuitry within the IMD. Connector module 50 is formed with a hole 53. A set screw 55 is disposed in hole 53 to tighten the lead in place and ensure electrical coupling between the lead and electrically conductive element 56.

A plug element 57 is positioned in hole 53 over set screw 55. Plug element 57 defines a slit 58, and a physician can insert a tool through slit 58 to tighten set screw 55. Upon removal of the tool from slit 58, slit 58 closes to provide a hermetic barrier to the interior of hole 53.

Connector module 50 further includes a retention element 59. In the example of FIG. 5, retention element 59 comprises a ring element inserted in hole 53. Retention element 59 defines an inner surface that conforms to the plug element 57 and an outer surface that presses against a wall 51 of hole 53 to create frictional forces between retention element 59 and the wall 51 of hole 53. Such a frictional force retention element 59 is sometimes referred to as a press-bush.

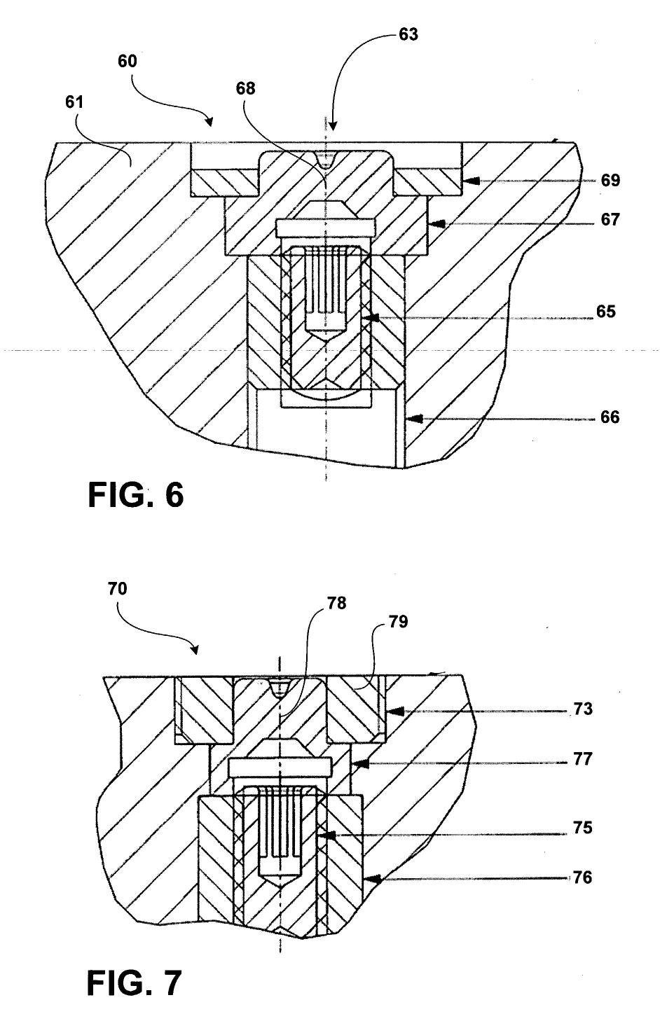

FIG. 6 is another cross sectional side view of a portion of a connector module 60, that may correspond to connector module 7 of FIG. 1. Connector module 60 is substantially similar to connector module 30, and includes a channel (not shown) sized to receive a proximal end of a medical lead (not shown). Upon insertion of the medical lead into the channel, the medical lead comes into electrical contact with electrically conductive element 66.

Connector module 60 is formed with a hole 63 and a set screw 65 is disposed in hole 63 to tighten the lead in place and ensure electrical coupling between the lead and electrically conductive element 66. A plug element 67 is positioned in hole 63 over set screw 65. In this example, plug element 67 is formed to mechanically interact with set screw 65, e.g., to fit around at least a portion of set screw 65. Plug element 67 defines a slit 68. A physician can insert a tool through slit 68 to tighten set screw 65. Upon removal of the tool from slit 68, however, slit 68 closes to provide a hermetic barrier to the interior of hole 63.

Connector module 60 further includes a retention element 69. In the example of FIG. 6, retention element 69 comprises a ring element that is ultrasonically welded over hole 63 to secure and retain plug element 67. For example, the retention element 69 may comprise a material that is the same as material 61 of connector module 60, e.g., a hard thermoplastic material. Ultrasonic welding generally melts retention element 69 to material 61. In this manner, fabrication of connector module 60 can be simplified and well suited for large scale production.

FIG. 7 is another cross sectional side view of a portion of a connector module 70, that may correspond to connector module 7 of FIG. 1. Connector module 70 includes a channel (not shown) sized to receive a proximal end of a medical lead (not shown). Upon insertion of the medical lead into the channel, the medical lead comes into electrical contact with electrically conductive element 76. Electrically conductive element, in turn electrically couples to circuitry of the IMD.

Connector module 70 is formed with a hole 73 and a set screw 75 is disposed in hole 73 to tighten the lead in place and ensure electrical coupling between the lead and electrically conductive element 76. A plug element 77 is positioned in hole 73 over set screw 75. In this example, plug element 77 is formed to mechanically interact with set screw 75, e.g., to fit over and around at least a portion of set screw 75. Plug element 77 defines a slit 78. A physician can insert a tool through slit 78 to tighten set screw 75. Upon removal of the tool from slit 78, however, slit 78 closes to provide a hermetic barrier to the interior of hole 73.

Connector module 70 further includes a retention element 79. In the example of FIG. 7, retention element 79 comprises a treaded ring element that threaded screws into hole 73. Treading 72 is also formed on the wall of hole 73 such that retention element 79 can screw into hole 73.

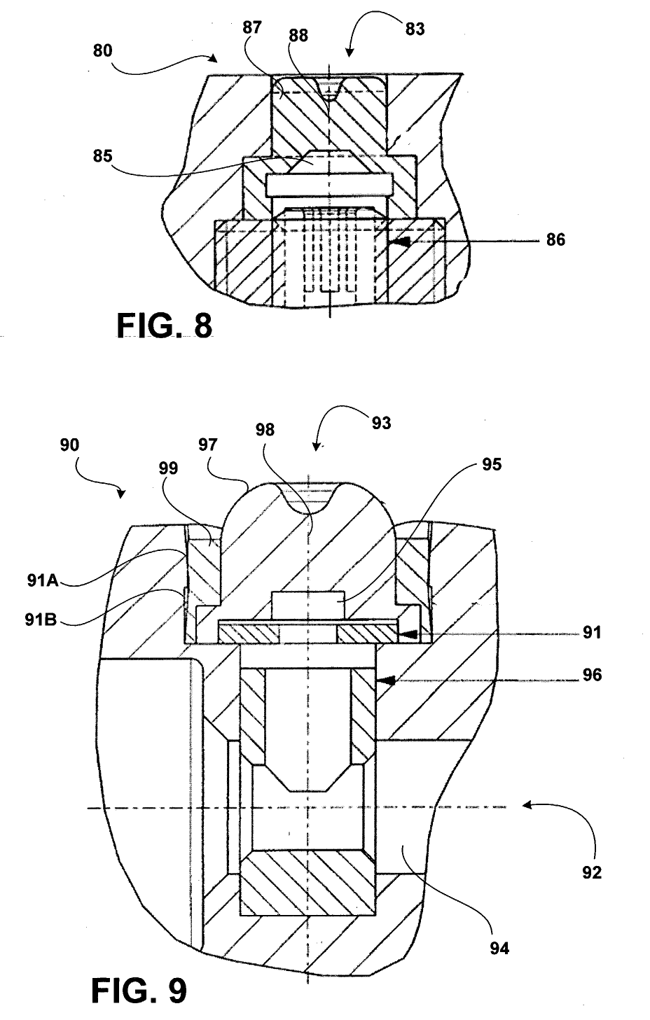

FIG. 8 is another cross sectional side view of a portion of a connector module 80, that may correspond to connector module 7 of FIG. 1. Connector module 80 includes a channel (not shown) sized to receive a proximal end of a medical lead (not shown). Upon insertion of the medical lead into the channel, the medical lead comes into electrical contact with conductive element 86.

Connector module 80 is formed with a hole 83 and a set screw 85 is disposed in hole 83 to tighten the lead in place and ensure electrical coupling between the lead and electrically conductive element 86. A plug element 87 is positioned in hole 83 over set screw 85. In this example, plug element 87 is formed to mechanically interact with set screw 85, e.g., to fit over and around at least a portion of set screw 85. Plug element 87 defines a slit 88. A physician can insert a tool through slit 88 to tighten set screw 85. Upon removal of the tool from slit 88, however, slit 88 closes to provide a hermetic barrier to the interior of hole 83.

Connector module 80 further includes a retention element 89. In the example of FIG. 8, retention element 89 comprises an integrally formed lip of connector module 80 that serves to hold plug element 87 in place. Furthermore, in this example, the various elements are formed such that plug element 87 can be inserted through the channel and into the bottom of hole 83. In this manner, assembly can be achieved when retention element 89 is integrally formed in connector module 80.

FIG. 9 is a cross sectional side view of a portion of a connector module 90, that may correspond to connector module 7 of FIG.1. Connector module 90 includes a channel 92 sized to receive a proximal end of a medical lead 94. An electrically conductive element 96 is disposed along channel 92 to electrically couple to medical lead 94. Connector module 90 is formed with a hole 93, e.g., perpendicular to channel 92. A set screw 95 is disposed in hole 93 to tighten lead 94 in place and ensure electrical coupling between lead 94 and electrically conductive element 96. Metal ring 91 improves mechanical coupling of set screw 95 within hole 93.

In accordance with the invention, a plug element 97 is positioned in hole 93 over set screw 95 and metal ring 91. Plug element 97 defines a slit 98. A physician can insert a tool through slit 98 to tighten set screw 95. Upon removal of the tool from slit 98, however, slit 98 closes to provide a hermetic barrier to the interior of hole 93.

Connector module 90 further includes a retention element 99. In the example of FIG. 9, retention element 99 comprises a ring element inserted in hole 93. Retention element 99 defines an inner surface that conforms to the plug element 97 and an outer surface that presses against a wall 91 of hole 93 to create frictional forces between retention element 99 and the wall 91 of hole 93. Moreover in this example, wall 91 of hole 93 defines variable widths that increase frictional forces between retention element 99 and wall 91 of the hole 93. Specifically, portion 91A of wall 91 is wider than potion 91B. Retention element 99 may flex outward in the area proximate to portion 91A such that frictional forces between retention element 99 and wall 91, specifically at the juncture of portions 91A and 91B, is enhanced to ensure that retention element 99 is secured in hole 93 to retain plug element 97.

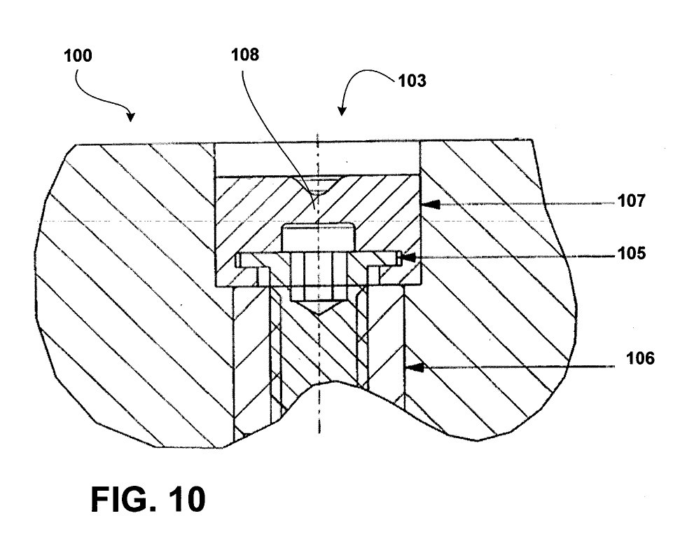

FIG. 10 is a cross sectional side view of a portion of a connector module 100, that may correspond to connector module 7 of FIG. 1. Connector module 100 includes a channel (not shown) sized to receive a proximal end of a medical lead (not shown). Upon insertion of the medical lead into the channel, the medical lead comes into electrical contact with electrically conductive element 106. Electrically conductive element 106, in turn, electrically couples to circuitry of the IMD.

In accordance with the invention, a plug element 107 is positioned in hole 103 over set screw 105. In this example, plug element 107 is formed to mechanically interact with set screw 105, e.g., to fit over and around set screw 105. Moreover, the mechanical interaction between set screw 105 and plug element 107 serves to hold plug element 107 within hole 103. Plug element 107 defines a slit 108. A physician can insert a tool through slit 108 to tighten set screw 105. Upon removal of the tool from slit 108, however, slit 108 closes to provide a hermetic barrier to the interior of hole 103.

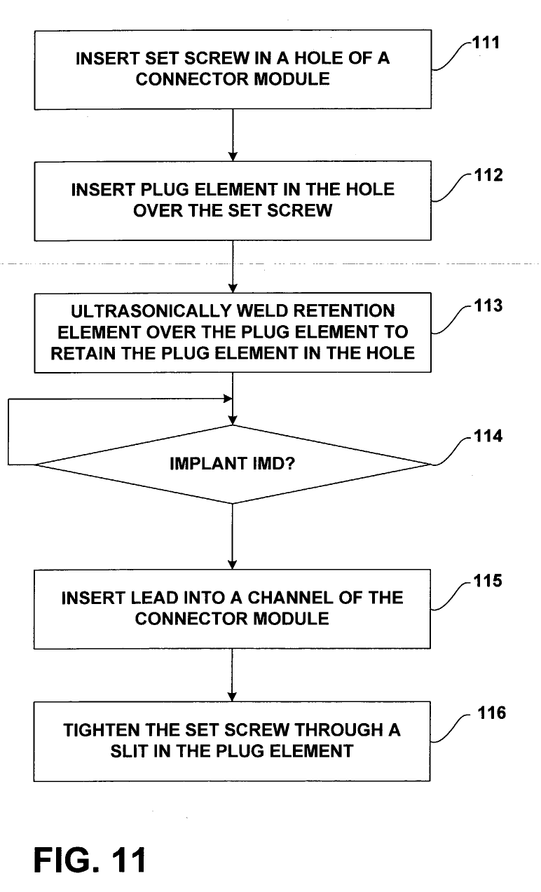

FIG. 11 is a flow diagram illustrating the assembly and use of an IMD according to an embodiment of the invention. In particular, FIG. 11 illustrates an assembly process which is highly advantageous for high volume manufacture of IMDs. As shown in FIG. 11, in order to assemble the IMD, a set screw 35 (FIG. 3) is inserted into a hole 33 of a connector module 30 (111). A plug element 37 is inserted in hole 33 over set screw 35 (112). Retention element 39 is then ultrasonically welded over plug element 37 to retain plug element 37 within hole 33 (133).

Following assembly, when the IMD is implanted in a patient (yes branch of 114), a physician inserts a proximal end of a medical 34 lead into a channel 32 of the connector module 30 of the IMD (115). The physician then tightens set screw 35 by inserting a tool through a slit 38 in plug element 37. In particular, the physician tightens set screw 35 to secure lead 34 in channel 32 and ensure electrical coupling between lead 34 and conductive element 36. Once set screw 35 is tightened through slit 38, slit 38 closes to ensure a hermetic seal. Retention element, 39 ensures that plug element 37 cannot be removed from hole 33.

A number of embodiments of the invention have been described. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable medical device comprising:
a housing;
circuitry within the housing; and
a connector module to receive a medical lead and electrically couple the medical lead to the circuitry, the connector module including:
a channel to receive the lead;
an electrical contact element coupled to the circuitry and disposed along the channel to electrically couple to the lead;
a hole formed in the connector module for access to the channel;
a set screw in the hole to hold the lead in place following insertion of the lead into the channel;
a plug element in the hole covering the set screw and formed with a slit that allows a tool to be inserted through the plug element to tighten the set screw; and
a retention element to retain the plug element in the connector module.

2. The implantable medical device of claim 1, wherein the retention element comprises a ring element ultrasonically welded to the connector module to define a lip over the hole.

3. The implantable medical device of claim 1, wherein the retention element comprises a lip integrally formed in the connector module over the hole.

4. The implantable medical device of claim 1, wherein the retention element comprises a ring element inserted in the hole, the ring element defining an inner surface that conforms to the plug element and an outer surface that presses against a wall of the hole to create frictional forces between the retention element and the wall of the hole.

5. The implantable medical device of claim 4, wherein the hole defines variable widths that increase frictional forces between the ring element and the wall of the hole.

6. The implantable medical device of claim 1, wherein the retention element comprises a threaded ring element that screws into the hole.

7. The implantable medical device of claim 1, wherein the hole is disposed perpendicular to the channel.

8. The implantable medical lead of claim 1, wherein the plug element is shaped to mechanically interact with the set screw.

9. The implantable medical lead of claim 1, further comprising a ring, wherein the set screw passes through the ring and the plug element covers the set screw and the ring.

10. The implantable medical lead of claim 1, wherein the connector module forms part of the housing.

11. A connector module of an implantable medical device to receive a medical lead and electrically couple the medical lead to circuitry of the implantable medical device, the connector module comprising:
a channel to receive the lead;
an electrical contact element coupled to the circuitry and disposed along the channel to electrically couple to the lead;
a hole formed in the connector module for access to the channel;
a set screw in the hole to hold the lead in place following insertion of the lead through the channel;
a plug element in the hole covering the set screw and formed with a slit that allows a tool to be inserted through the plug element to tighten the set screw; and
a retention element to retain the plug element in the connector module.

12. The connector module of claim 11, wherein the retention element comprises a ring element ultrasonically welded to the connector module over the hole.

13. The connector module of claim 11, wherein the retention element comprises a lip formed in the connector module over the hole.

14. The connector module of claim 11, wherein the retention element comprises a ring element inserted in the hole, the ring element defining an inner surface that conforms to the plug element and an outer surface that presses against a wall of the hole to create frictional forces between the retention element and the wall of the hole.

15. The connector module of claim 11, wherein the hole defines variable widths that increase frictional forces between the ring element and the wall of the hole.

16. The connector module of claim 11, wherein the retention element comprises a threaded ring element that screws into the hole.

17. The connector module of claim 11, wherein the hole is disposed perpendicular to the channel.

18. The connector module of claim 11, wherein the plug element is shaped to mechanically interact with the set screw.

19. The connector module of claim 11, further comprising ring, wherein the set screw passes through the ring and the plug element covers the set screw and the ring.

20. An implantable medical device comprising:
a housing;
circuitry within the housing; and
a connector module to receive a medical lead and electrically couple the medical lead to the circuitry, the connector module including:
a channel to receive the lead;
an electrical contact element coupled to the circuitry and disposed along the channel to electrically couple to the lead;
a hole formed in the connector module for access to the channel;
a set screw in the hole to hold the lead in place following insertion of the lead through the channel;
a plug element in the hole covering the set screw and formed with a slit that allows a tool to be inserted through the plug element to tighten the set screw; and
means for retaining the plug element in the connector module without the use of an adhesive.

21. The implantable medical device of claim 20, wherein the means for retaining the plug element comprises a ring element ultrasonically welded to the connector module over the hole.

22. The implantable medical lead of claim 21, further comprising a ring, wherein the set screw passes through the ring and the plug element covers the set screw and the ring.

23. A method comprising:
inserting a set screw in a hole of a connector module of an implantable medical device such that the set screw can be tightened to secure a lead in a channel of the connector module;
inserting a plug in the hole over the set screw, the plug being formed with a slit to allow access to the set screw; and
ultrasonically welding a retention element over the plug to retain the plug in the hole.

24. The method of claim 23, further comprising:
inserting a lead into the channel; and
tightening the set screw through the slit in the plug to secure the lead in the channel.
